(19) United States
(12) Patent Application Publication
(10) Pub. No.: US 2024/0269086 A1
(43) Pub. Date: Aug. 15, 2024

(54) PERGULARIA TOMENTOSA SILVER NANOPARTICLES

(71) Applicant: KING FAISAL UNIVERSITY, AL-AHSA (SA)

(72) Inventor: MUNIRAH FAHAD ALDAYEL, AL-AHSA (SA)

(21) Appl. No.: 18/108,368

(22) Filed: Feb. 10, 2023

(51) Int. Cl. A61K 9/51; A61K 36/24; A61P 31/04

(52) U.S. Cl. CPC A61K 9/5192; A61K 36/24; A61P 31/04; B82Y 5/00; B82Y 30/00; B82Y 40/00

(57) ABSTRACT

Pergularia tomentosa silver nanoparticles can have a crystalline structure and an average particle size ranging from about 5 nm to about 50 nm. The Pergularia tomentosa silver nanoparticles may be synthesized by providing a Pergularia tomentosa extract and combining the Pergularia tomentosa extract with 1 mM of silver nitrate. The extract may be synthesized by harvesting Pergularia tomentosa leaves, drying the Pergularia tomentosa leaves, powdering the dried Pergularia tomentosa leaves, mixing the powdered Pergularia tomentosa leaves with ethanol to provide a solution, extracting the solution to provide a plant extract, adding silver nitrate to the plant extract to provide a mixture, and microwaving the mixture to provide the Pergularia tomentosa silver nanoparticles.

### BACKGROUND

### 1. Field

The disclosure of the present patent application relates to silver nanoparticles, and particularly, to silver nanoparticles synthesized by bio-reduction of Pergularia tomentosa extract.

### 2. Description of the Related Art

Recently, nanoparticles have demonstrated important uses in a variety of fields. Nanoparticles have been used in electronics, sensing, optics, and medicine, for example.

Synthesis of nanoparticles has been achieved by a variety of methods, including physicochemical, thermal decomposition, electrochemical, microwave assisted, sonochemical, solvothermal, photosynthesis, photochemical reduction, chemical reduction and continuous-flow methods. These methods are often costly or produce by-products that pose increased risks to human health and the environment.

In recent years, green or environmentally friendly chemical methods have been developed to prepare nanoparticles using plant extracts. Green chemistry has the advantage of being safer, faster, environmentally friendly, and economical. However, the rise of green methods of preparing nanoparticles has also demonstrated that the activities and characteristics of the nanoparticles vary significantly, depending upon the detailed method of synthesis and specific plant extract used.

Pergularia tomentosa is a desert plant belonging to the Asclepiadacea family. The plant has been used medically for its anti-inflammatory properties, as well as other therapeutic properties.

Thus, nanoparticles synthesized using an environmentally friendly method solving the aforementioned problems are desired.

### SUMMARY

The present subject matter relates to Pergularia tomentosa silver nanoparticles. The Pergularia tomentosa silver nanoparticles can have a hexagonal shape and a crystalline structure. In an embodiment, the Pergularia tomentosa silver nanoparticles can have an average particle size ranging from about 5 nm to about 50 nm.

In an embodiment, the Pergularia tomentosa silver nanoparticles may be synthesized by providing a Pergularia tomentosa extract and combining the Pergularia tomentosa extract with 1 mM of silver nitrate. The extract may be synthesized by harvesting Pergularia tomentosa leaves, drying the Pergularia tomentosa leaves, powdering the dried Pergularia tomentosa leaves, mixing the powdered Pergularia tomentosa leaves with ethanol to provide a solution, and extracting the solution to provide the extract. In an embodiment, the extract may be combined with silver nitrate to provide a mixture and the mixture can be microwaved to facilitate bio-reduction. In an embodiment, the Pergularia tomentosa leaves may be harvested from Pergularia tomentosa plants grown in Al-Ahsa-Dammam Road, Saudi Arabía. In an embodiment the powdered Pergularia tomentosa leaves may be mixed with an appropriate solvent, including but not limited to ethanol.

According to an embodiment, a method of inhibiting bacterial growth can include administering an effective amount of the Pergularia tomentosa silver nanoparticles to a subject in need thereof.

According to an embodiment, a method of treating inflammation can include administering an effective amount of the Pergularia tomentosa silver nanoparticles to a subject in need thereof.

These and other features of the present subject matter will become readily apparent upon further review of the following specification and drawings.

### BRIEF DESCRIPTION OF THE DRAWINGS

FIGS. 1A-IF depict (FIG. 1A) UV-Vis spectrum of PT-AgNPs; (FIG. 1B) X-ray diffraction (XRD) analysis of PT-AgNPs revealing peaks at 20 values of 38.25°, 46.37°, 64.60°, and 77.62°; (FIG. 1C) fourier transform infrared (FTIR) spectrum of PT-AgNPs; (FIG. 1D) transmission electron microscopy (TEM) image showing the PT-AgNPs formed with different shapes, including hexagonal and spherical structures; (FIG. 1E) Energy-dispersive X-ray (EDX) spectrum of PT-AgNPS; and (FIG. 1F) Zeta potential of PT-AgNPs.

FIG. 2 is a graph showing time-kill curve plots of S. enteritidis, after exposure to the PT-AgNPs.

FIGS. 3A-3B show the effect of PT-AgNPs on leakage of reducing sugar (FIG. 3A) and proteins (FIG. 3B) from S. epidermidis (different letters indicate significant differences (p<0.05) according to Duncan's test).

FIG. 4A shows DPPH radical scavenging activity from different concentrations of PT-AgNPs synthesized using P. tomentosa leaf extract.

FIG. 4B shows anti-inflammatory activity of the PT-AgNPs (different letters indicate significant differences (p<0.05) according to Duncan's test).

FIG. 5 is a graph showing the effect of PT-AgNPs on the MCF-7 cell line.

Similar reference characters denote corresponding features consistently throughout the attached drawings.

### DETAILED DESCRIPTION OF THE PREFERRED EMBODIMENTS

The following definitions are provided for the purpose of understanding the present subject matter and for construing the appended patent claims.

### Definitions

It should be understood that the drawings described above or below are for illustration purposes only. The drawings are not necessarily to scale, with emphasis generally being placed upon illustrating the principles of the present teachings. The drawings are not intended to limit the scope of the present teachings in any way.

Throughout the application, where compositions are described as having, including, or comprising specific components, or where processes are described as having, including, or comprising specific process steps, it is contemplated that compositions of the present teachings can also consist essentially of, or consist of, the recited components, and that the processes of the present teachings can also consist essentially of, or consist of, the recited process steps.

It is noted that, as used in this specification and the appended claims, the singular forms “a”, “an”, and “the” include plural references unless the context clearly dictates otherwise.

In the application, where an element or component is said to be included in and/or selected from a list of recited elements or components, it should be understood that the element or component can be any one of the recited elements or components, or the element or component can be selected from a group consisting of two or more of the recited elements or components. Further, it should be understood that elements and/or features of a composition or a method described herein can be combined in a variety of ways without departing from the spirit and scope of the present teachings, whether explicit or implicit herein.

The use of the terms “include,” “includes”, “including,” “have,” “has,” or “having” should be generally understood as open-ended and non-limiting unless specifically stated otherwise.

A “subject” herein is typically a human. In certain embodiments, a subject is a non-human mammal. Exemplary non-human mammals include laboratory, domestic, pet, sport, and stock animals, e.g., mice, cats, dogs, horses, and cows. As used herein, the term “patient” refers to any single subject for which treatment is desired. In certain embodiments, the patient herein is a human. A subject can be considered to be in need of treatment.

The use of the singular herein includes the plural (and vice versa) unless specifically stated otherwise. In addition, where the use of the term “about” is before a quantitative value, the present teachings also include the specific quantitative value itself, unless specifically stated otherwise. As used herein, the term “about” refers to a ±10% variation from the nominal value unless otherwise indicated or inferred.

Unless defined otherwise, all technical and scientific terms used herein have the same meaning as commonly understood to one of ordinary skill in the art to which the presently described subject matter pertains.

Where a range of values is provided, for example, concentration ranges, percentage ranges, or ratio ranges, it is understood that each intervening value, to the tenth of the unit of the lower limit, unless the context clearly dictates otherwise, between the upper and lower limit of that range and any other stated or intervening value in that stated range, is encompassed within the described subject matter. The upper and lower limits of these smaller ranges may independently be included in the smaller ranges, and such embodiments are also encompassed within the described subject matter, subject to any specifically excluded limit in the stated range. Where the stated range includes one or both of the limits, ranges excluding either or both of those included limits are also included in the described subject matter.

Throughout the application, descriptions of various embodiments use “comprising” language. However, it will be understood by one of skill in the art, that in some specific instances, an embodiment can alternatively be described using the language “consisting essentially of” or “consisting of”.

In an embodiment, the present subject matter relates to Pergularia tomentosa silver nanoparticles (PT-AgNPs). The Pergularia tomentosa silver nanoparticles may be synthesized by providing a Pergularia tomentosa extract and combining the Pergularia tomentosa extract with 1 mM of silver nitrate. In one embodiment, the extract may be synthesized by harvesting Pergularia tomentosa leaves, drying the Pergularia tomentosa leaves, powdering the dried Pergularia tomentosa leaves, mixing the powdered Pergularia tomentosa leaves with a solvent to provide a solution, and extracting the solution to provide a plant extract. The silver nitrate can be added to the plant extract to provide a mixture including the Pergularia tomentosa silver nanoparticles. In an embodiment, the mixture can be heated, e.g., microwaved, to complete the bio-reduction process. In an embodiment, the Pergularia tomentosa leaves may be harvested from Pergularia tomentosa plants grown in Al-Ahsa-Dammam Road, Al-Ahsa, Saudi Arabia. In an embodiment, the powdered Pergularia tomentosa leaves may be mixed with an appropriate solvent, including but not limited to ethanol.

In an embodiment, the plant leaves can be air dried and then ground into a plant powder. In an embodiment, about 5 grams of the plant powder can be dissolved in about 50 mL of ethanol and extracted using a Soxhlet extractor at about 50° ° C. to provide a plant extract. In an embodiment, about 1 mM of silver nitrate can be added to the plant extract to provide a mixture. The silver nitrate can reduce the plant extract into Ag+ ions. In an embodiment, the mixture can be microwaved to complete the bio-reduction process and provide the Pergularia tomentosa silver nanoparticles. For example, the mixture can be microwaved for a period of time ranging from about 3 minutes to about 5 minutes. In an embodiment, the mixture is microwaved for four minutes. A color change in the mixture to colloidal brown can indicate synthesis of the nanoparticles.

In an embodiment, the Pergularia tomentosa silver nanoparticles can have a hexagonal shape and a crystalline structure. The Pergularia tomentosa silver nanoparticles can have an average particle size ranging from about 5 nm to about 50 nm or from about 10 nm to about 45 nm, for example, about 10 nm, about 20 nm, about 30 nm, about 40 nm, about 50 nm, or any range of any two endpoints thereof.

An embodiment of the present subject matter is directed to a pharmaceutical composition comprising the Pergularia tomentosa silver nanoparticles and a pharmaceutically acceptable carrier.

An embodiment of the present subject matter is directed to a method of making a pharmaceutical composition including mixing the Pergularia tomentosa silver nanoparticles with a pharmaceutically acceptable carrier. For example, the method of making a pharmaceutical composition can include mixing the Pergularia tomentosa silver nanoparticles under sterile conditions with a pharmaceutically acceptable carrier with preservatives, buffers, and/or propellants to create the pharmaceutical composition.

To prepare the pharmaceutical composition, the Pergularia tomentosa silver nanoparticles, as the active ingredient, are intimately admixed with a pharmaceutically acceptable carrier according to conventional pharmaceutical compounding techniques. Carriers are inert pharmaceutical excipients, including, but not limited to, binders, suspending agents, lubricants, flavorings, sweeteners, preservatives, dyes, and coatings. In preparing compositions in oral dosage form, any of the pharmaceutical carriers known in the art may be employed. For example, for liquid oral preparations, suitable carriers and additives include water, glycols, oils, alcohols, flavoring agents, preservatives, coloring agents, and the like. Further, for solid oral preparations, suitable carriers and additives include starches, sugars, diluents, granulating agents, lubricants, binders, disintegrating agents, and the like.

The present compositions can be in unit dosage forms such as tablets, pills, capsules, powders, granules, ointments, sterile parenteral solutions or suspensions, metered aerosol or liquid sprays, drops, ampules, auto-injector devices or suppositories, for oral parenteral, intranasal, sublingual or rectal administration, or for administration by inhalation or insufflation. The Pergularia tomentosa silver nanoparticles can be mixed under sterile conditions with a pharmaceutically acceptable carrier and, if required, any needed preservatives, buffers, or propellants. The composition can be presented in a form suitable for daily, weekly, or monthly administration. The pharmaceutical compositions herein will contain, per dosage unit, e.g., tablet, capsule, powder, injection, teaspoonful, suppository and the like, an amount of the active ingredient necessary to deliver an effective dose. A therapeutically effective amount of the Pergularia tomentosa silver nanoparticles or an amount effective to treat a disease, such as a bacterial infection, may be determined initially from the Examples described herein and adjusted for specific targeted diseases using routine methods.

The Pergularia tomentosa silver nanoparticles can have antibacterial, antioxidant, and anti-inflammatory properties. The Pergularia tomentosa silver nanoparticles can be administered to a subject in need thereof. In an embodiment, the Pergularia tomentosa silver nanoparticles can be administered to a subject in need thereof to inhibit bacterial growth. In an embodiment, the Pergularia tomentosa silver nanoparticles can be administered to a subject to inhibit the growth of at least one bacteria selected from the group consisting of S. aureus, S. enteritidis, E. coli, and B. cereus. In a further embodiment, the Pergularia tomentosa silver nanoparticles can be administered to a subject in need thereof to treat inflammation.

An embodiment of the present subject matter is directed to a method of inhibiting bacterial growth in a subject, comprising administering to a subject in need thereof a therapeutically effective amount of the pharmaceutical composition according to the present subject matter.

An embodiment of the present subject matter is directed to a method of treating inflammation in a subject, comprising administering to a subject in need thereof a therapeutically effective amount of the pharmaceutical composition according to the present subject matter.

The Pergularia tomentosa silver nanoparticles or pharmaceutical compositions thereof can be administered to a subject by any suitable route. For example, the compositions can be administered orally (including bucally and sublingually), nasally, rectally, intracisternally, intra vaginally, intraperitoneally, topically, transdermally (as by powders, ointments, or drops), and/or parenterally. As used herein, “parenteral” administration refers to modes of administration other than through the gastrointestinal tract, which include intravenous, intramuscular, intraperitoneal, intrasternal, intramammary, intraocular, retrobulbar, intrapulmonary, intrathecal, subcutaneous and intraarticular injection and infusion. Surgical implantation may also be contemplated, including, for example, embedding a composition of the disclosure in the body such as, for example, in a tissue, in the abdominal cavity, under the splenic capsule, brain, or in the cornea.

The present teachings are illustrated by the following examples.

### Example 1

### Pergularia tomentosa Silver Nanoparticles

Pergularia tomentosa leaves were gathered from Al-Ahsa-Dammam Road, Saudi Arabia. The plant leaves were air dried and then reduced to a plant powder. 5 g of the plant powder was dissolved in 50 mL of ethanol and extracted using a Soxhlet extractor at 50° C.

1 mM of silver nitrate was added to the extract to reduce the plant extract into Ag+ ions. The resulting mixture was microwaved for four minutes to accelerate the bio-reduction process. The color of the mixture changed to a colloidal brown, indicating synthesis of Pergularia tomentosa silver nanoparticles (PT-AgNPs).

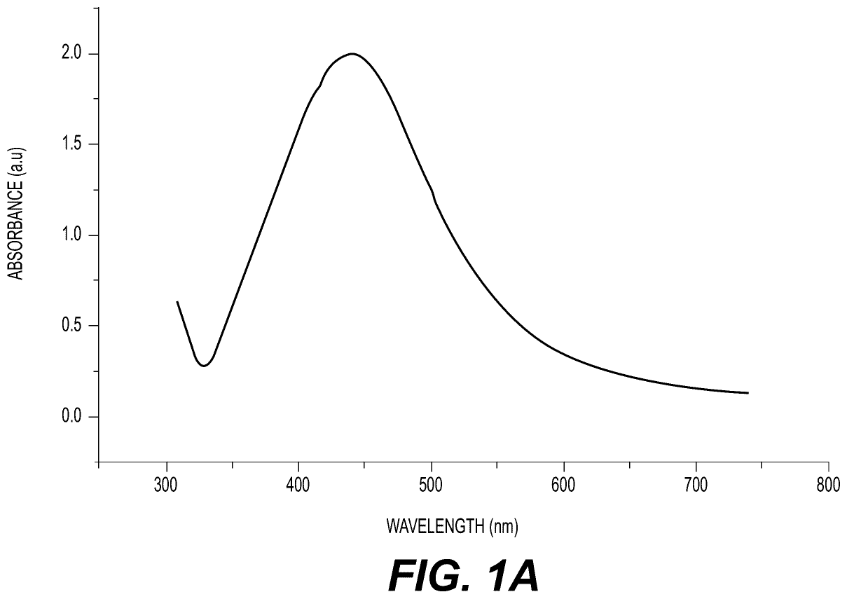

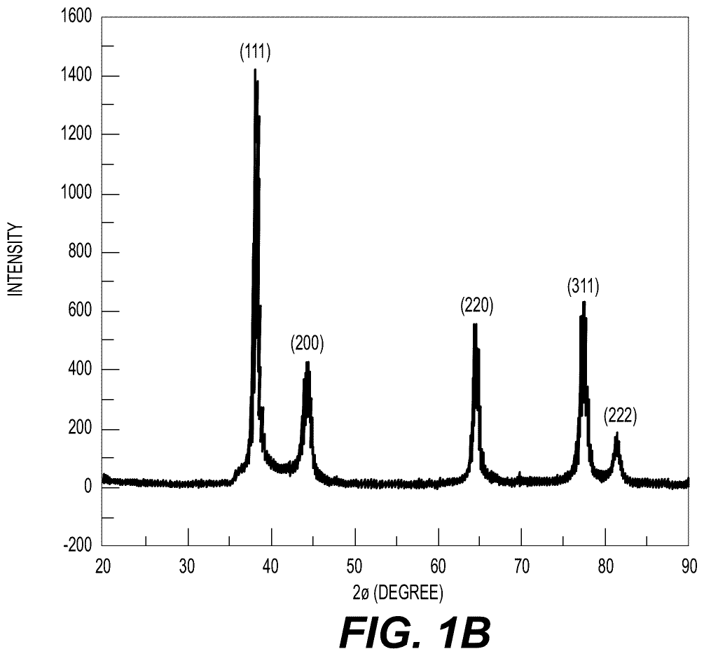

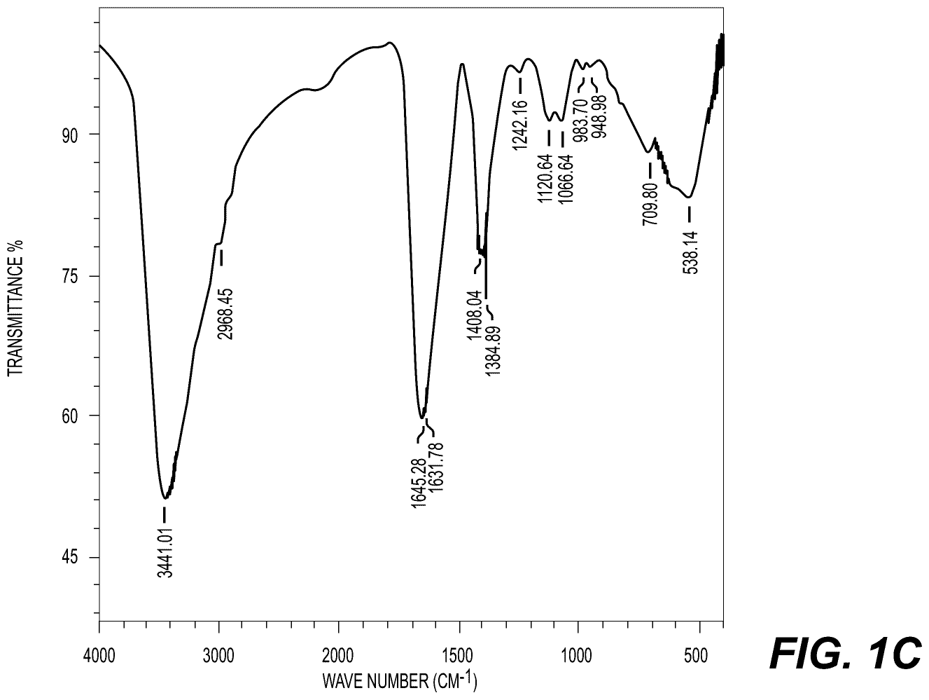

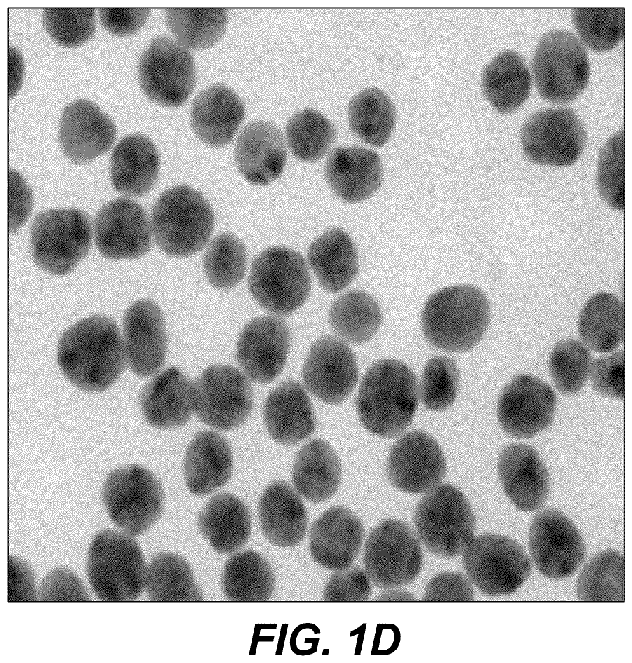

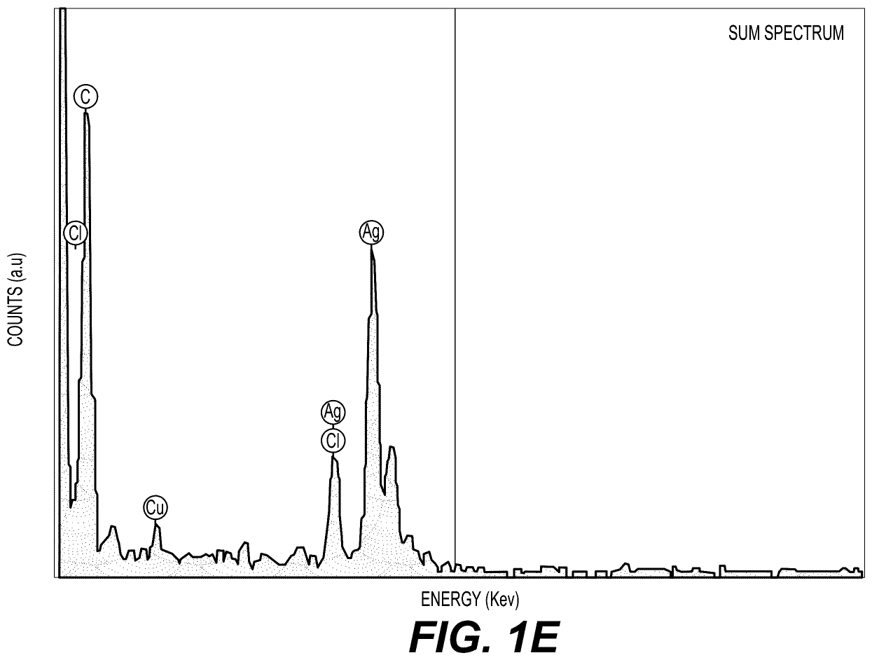

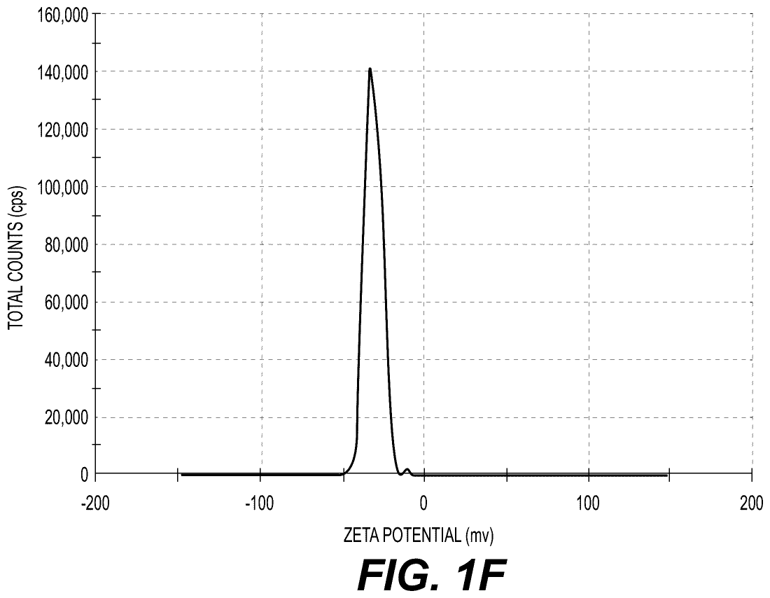

The PT-AgNPs were characterized. FIGS. 1A-IF depict (FIG. 1A) UV-Vis spectrum of PT-AgNPs; (FIG. 1B) X-ray diffraction (XRD) analysis of PT-AgNPs revealing peaks at 20 values of 38.25°, 46.37°, 64.60°, and 77.62°; (FIG. 1C) fourier transform infrared (FTIR) spectrum of PT-AgNPs; (FIG. 1D) transmission electron microscopy (TEM) image shows the PT-AgNPs formed with different shapes, including hexagonal and spherical structures; (FIG. 1E) Energy-dispersive X-ray (EDX) spectrum of PT-AgNPS; and (FIG. 1F) Zeta potential of PT-AgNPs.

The results showed that PT-AgNPs had a hexagonal and crystalline structure with an average size of 5 nm-50 nm.

### Example 2

### Pergularia tomentosa Silver Nanoparticles Activity

The antioxidant, antibacterial, and anti-inflammatory activities of the green-synthesized AgNPs (PT-AgNPs) were investigated.

The agar disk diffusion method was used to investigate the antibacterial activity of the PT-AgNPs against four bacterial strains. The AgNPs exhibited antibacterial activity on the tested bacterial species with an MIC range of 50 μg/mL-100 μg/mL.

Table I shows antibacterial potential of PT-AgNPs against some pathogenic bacteria.

TABLE 1
Antibacterial Potential of PT-AgNPs against some Pathogenic Bacteria

| | Antibacterial agent | | | | | | | | | | | | | | | | | | | |
|---|---|---|---|---|---|---|---|---|---|---|---|---|---|---|---|---|---|---|---|---|
| | DMSO | | | | | | | PT-AgNPs | | | | | | | Chloramphenicol | | | | | |
| | Concentration (μg/ml) | | | | | | | | | | | | | | | | | | | |
| Pathogenic | 0 | 6.25 | 12.5 | 25 | 50 | 100 | 200 | 0 | 6.25 | 12.5 | 25 | 50 | 100 | 200 | 0 | 6.25 | 12.5 | 25 | 50 | 100 |

| bacteria | IZD (mm) | | | | | | | | | | | | | | | | | |
|---|---|---|---|---|---|---|---|---|---|---|---|---|---|---|---|---|---|---|
| S. aureus | [symbol] | [symbol] | [symbol] | [symbol] | [symbol] | [symbol] | [symbol] | [symbol] | [symbol] | [symbol] | [symbol] | [symbol] | [symbol] | Nil | [symbol] | [symbol] | [symbol] | Nil |
| S. enteritidis | [symbol] | [symbol] | [symbol] | [symbol] | [symbol] | [symbol] | [symbol] | [symbol] | [symbol] | [symbol] | [symbol] | [symbol] | Nil | | [symbol] | [symbol] | Nil | |
| E. coli | [symbol] | [symbol] | [symbol] | [symbol] | [symbol] | [symbol] | [symbol] | [symbol] | [symbol] | [symbol] | [symbol] | [symbol] | [symbol] | Nil | [symbol] | [symbol] | [symbol] | Nil |
| B. cereus | [symbol] | [symbol] | [symbol] | [symbol] | [symbol] | [symbol] | [symbol] | [symbol] | [symbol] | [symbol] | [symbol] | [symbol] | [symbol] | Nil | [symbol] | [symbol] | [symbol] | Nil |

IZD = Inhibition zone diameter (mm)
Nil = No growth [symbol]
indicates data missing or illegible when filed

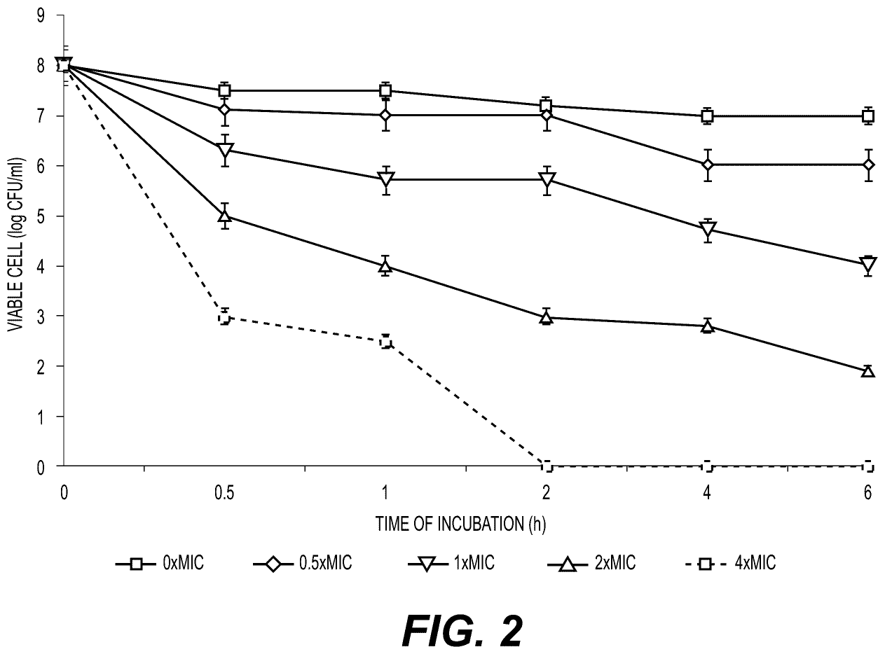

FIG. 2 shows the time-kill curve plots of S. enteritidis, after exposure to the PT-AgNPs.

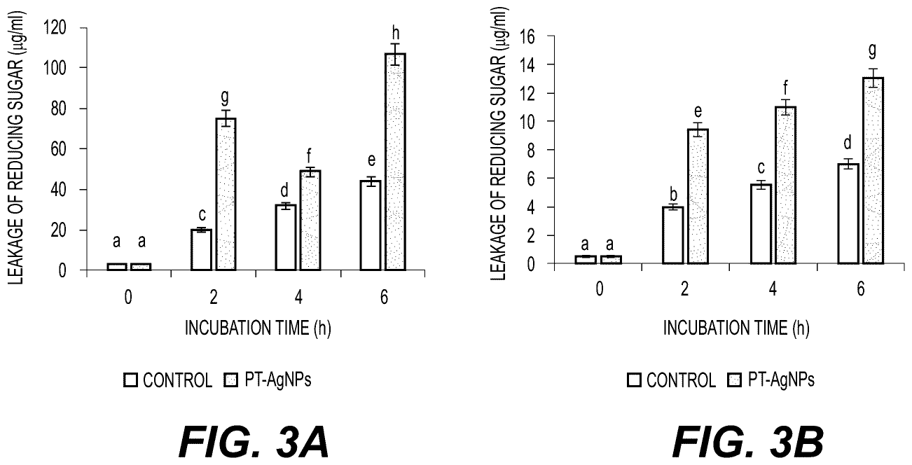

Different morphological modifications could be detected after treating the bacteria with PT-AgNPs, for example cells treated with PT-AgNPs (50 μg/mL), showed that the cells formed septa and spheroplasts. FIGS. 3A-3B show the effect of PT-AgNPs on leakage of reducing sugar (FIG. 3A) and proteins (FIG. 3B) from S. epidermidis (different letters indicate significant differences (p<0.05) according to Duncan's test).

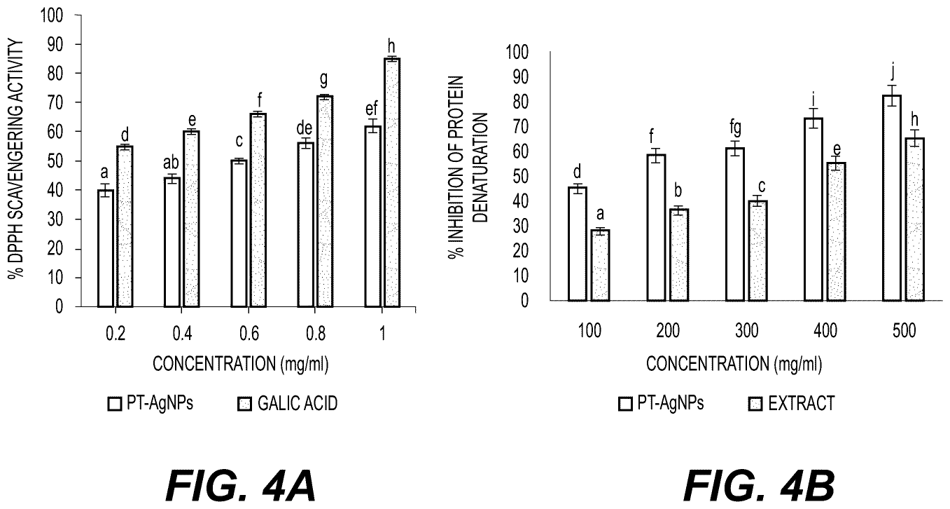

The antioxidant and anti-inflammatory activities of PT-AgNPs are summarized in FIGS. 4A-4B. FIG. 4A shows DPPH radical scavenging activity from different concentrations of PT-AgNPs synthesized using P. tomentosa leaf extract. FIG. 4B shows anti-inflammatory activity of the PT-AgNPs (different letters indicate significant differences (p<0.05) according to Duncan's test). Interestingly, PT-AgNPs displayed increased anti-inflammatory activity as compared to the P. tomentosa leaf extract (79% versus 59% at 500 μg/mL). The PTAgNPs displayed radical scavenging activity with an IC50 of 0.69 mg/mL.

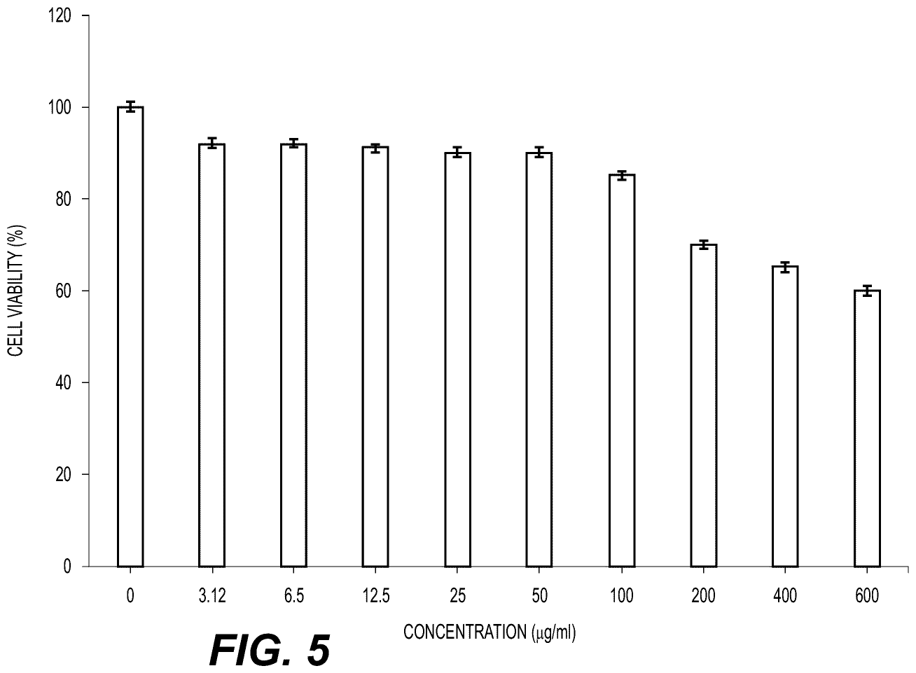

Cytotoxicity of PT-AgNPs on the MCF-7 cell line was also studied. The dose-dependent effect of PT-AgNPs evaluated using an MTT assay after 24 h of treatment is summarized in FIG. 5. As shown in FIG. 5, the PT-AgNPs did not display any cytotoxicity in the MCF-7 cell line at MIC concentration.

Accordingly, it was determined that PT-AgNPs exhibited useful antioxidant, antibacterial, and anti-inflammatory properties and could safely be used in several medical applications.

It is to be understood that the Pergularia tomentosa silver nanoparticles are not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

1. Pergularia tomentosa silver nanoparticles comprising nanoparticles synthesized from Pergularia tomentosa extract and silver nanoparticles.

2. The Pergularia tomentosa silver nanoparticles of claim 1, wherein the nanoparticles have an average particle diameter of about 5 nm to about 50 nm.

3. The Pergularia tomentosa silver nanoparticles of claim 1, wherein the nanoparticles have an average particle diameter of about 10 nm to about 45 nm.

4. A pharmaceutical composition, comprising the Pergularia tomentosa silver nanoparticles of claim 1 and a pharmaceutically acceptable carrier.

5.-10. (canceled)

11. Pergularia tomentosa silver nanoparticles prepared by a method comprising:
mixing powdered Pergularia tomentosa leaves with a solvent to form a solution;
extracting the solution to provide a plant extract; and
adding silver nitrate to the plant extract to provide a mixture including silver nanoparticles.

12. The Pergularia tomentosa silver nanoparticles of claim 11, wherein the nanoparticles have an average particle diameter of about 5 nm to about 50 nm.

13. The Pergularia tomentosa silver nanoparticles of claim 11, wherein the nanoparticles have an average particle diameter of about 10 nm to about 45 nm.

14.-16. (canceled)
